(19) United States
(12) Patent Application Publication
(10) Pub. No.: US 2022/0072050 A1
(43) Pub. Date: Mar. 10, 2022

(54) Dual Stem Cell Therapy for Neurological Conditions

(71) Applicant: Abraham J and Phyllis Katz Cord Blood Foundation, Cleveland, OH (US)

(72) Inventors: Mary Laughlin, Cleveland, OH (US); Daniel Zwick, Cleveland, OH (US)

(21) Appl. No.: 17/417,289

(22) PCT Filed: Jan. 17, 2020
(86) PCT No.: PCT/US20/14041

§ 371 Date: Jun. 22, 2021

Related U.S. Application Data
(60) Provisional application No. 62/794,041, filed on Jan. 18, 2019.

(51) Int. Cl. A61K 35/28; C12N 5/0775; C12N 5/0789

(52) U.S. Cl. CPC A61K 35/28; C12N 5/0665; C12N 5/0647; C12N 2533/32; C12N 2502/137; C12N 2501/599; C12N 2502/1171

(57) ABSTRACT

Compositions and methods for treating neuronal injury, such as in Parkinson's disease, comprising administration of hematopoietic stem cells and mesenchymal stromal cells to a subject are provided. Methods for producing such compositions from blood, including umbilical cord blood are also provided.

### CROSS REFERENCE TO RELATED APPLICATIONS

This application claims the priority benefit of U.S. Provisional Application No. 62/794,041 filed Jan. 18, 2019, which application is incorporated herein by reference.

### FIELD OF THE INVENTION

The present disclosure relates generally to cellular compositions and methods for treating Parkinson's disease and other neurological conditions.

### BACKGROUND

Parkinson's disease (PD) is a debilitating neurodegenerative disease affecting nearly 1 million Americans.^{1,2} PD involves the gradual degeneration of neurons in the Substantia Nigra (SN) that produce dopamine, which ultimately results in debilitating motor deficits and depression. At present, the standard drug treatment regimen of carbidopa/levodopa (Sinemet) can enhance endogenous dopamine release and alleviate PD symptoms, yet patients who are treated with levodopa long term may experience dyskinesia at some point, usually three to five years after starting the medication. Most importantly, levodopa does not induce the regeneration of dopaminergic neurons, and existing therapies have limited efficacy in controlling or reversing disease progression long term.³

There is a need to develop innovative therapeutic approaches to PD. Stem cells offer a promising means to control and potentially reverse disease pathogenesis. The potential of stem cells in PD therapy has been recognized for more than 3 decades after early proof of concept human intervention studies using allogeneic fetal tissue transplantation were shown to reverse symptoms and PD pathology. Post-mortem analysis of these PD patients suggested that allogeneic fetal dopaminergic tissue grafts appeared to be capable of incorporating anatomically and forming normal synaptic contacts with host striatal projection neurons.⁴ Yet both direct and indirect mechanisms appear to underlie the therapeutic effects of stem cell therapy.⁵ Human embryonic stem cells (hESC) and induced pluripotent stem cells (iPSC) in pre-clinical studies have shown the potential to differentiate into and replace dopaminergic neurons, however hESC carry ethical concerns, and both sources of stem cells carry the risk of teratoma formation.

In contrast, umbilical cord blood (UCB) is a rich source of primitive stem cells that is safer and more readily acquired than hESC and iPSC. There is no invasive procedure or genetic manipulation with oncogenes required, or ethical concerns associated with UCB stem cells, and the current global inventory of >660,000 clinical grade UCB stem cell grafts, linked to transplant physician teams via web-based search engines, ensures optimal HLA match and cell dose for individual patients requiring stem cell transplantation.⁶ UCB is approved by all religious groups including the Vatican.⁷ In addition, long-term (>30 year) observations of the approximately 40,000 humans treated with UCB stem cell therapy, reveals that no patient has any evidence of donor derived teratoma formation.

Mesenchymal stromal cells (MSCs) have shown neuro-regenerative potential in animal models and clinical trials.^{8,9} Unfortunately, MSC have not to date demonstrated control or reversal of PD clinical symptoms.¹⁰ The relative failure of MSCs in human PD trials has been unexpected, given evident neuroprotective capacity in pre-clinical in vitro and in murine PD pre-clinical models. As PD particularly affects the elderly/individuals often with concurrent reduced micro-vasculogenesis, one limitation of MSC therapy alone is that this stem cell population has only limited vasculogenic effects compared with hematopoietic stem cells (HSCs). PD pathophysiology is increasingly recognized to be attributable in part to microvascular degeneration. It has been previously observed that UCB derived CD133+ HSCs elicit robust vasculogenesis in vitro and in vivo.¹¹⁻¹⁹ Furthermore, UCB CD133+ HSCs have been shown to exert equivalent neuroprotective capacity when compared to MSC, including vasculogenic potential in rodent PD models.^{20,21} In limited circumstances, MSC/HSC were co-transplanted in hematopoietic stem cell transplantation.²⁵

In certain neurological treatment procedures deep brain stimulation (DBS) has been employed combining cell or tissue implantation with DBS.²² Patients undergoing DBS surgery can receive stereotactic intracranial administered stem cell graft simultaneously during the same surgical procedure.^{23,24} However, PD is increasingly recognized as a disease of the vasculature, particularly in the elderly, for whom DBS alone is generally least effective.²⁶⁻²⁸

Therefore, new therapeutic compositions, methods for their manufacture, and methods of treatment therewith for PD and other neurological conditions are needed.

### SUMMARY OF THE INVENTION

In embodiments, the present invention provides a novel stem cell therapeutic approach to neurological conditions, such as Parkinson's disease, based on identifying the complimentary and additive neuro-regenerative capacity of two separate populations of regenerative stem cells in UCB with utility in PD therapy comprising mesenchymal stromal cells (MSCs) and primitive CD133 expressing hematopoietic stem cells (HSCs).

In embodiments, the invention provides methods for treating injured neurons in a subject in need comprising administering to the subject a treatment effective amount of a first composition of substantially purified CD133+ HSCs and a second composition of substantially purified MSCs. In embodiments, the first and second compositions are combined prior to administration.

In embodiments, the HSCs and MSCs are substantially purified from blood, which may be autologous or allogeneic. In embodiments, the HSCs and MSCs are obtained from human umbilical cord blood, blood or bone marrow. In embodiments, the substantially purified compositions are isolated from at least 60%, 70%, 80%, 90% or 95% or more of the constituents found naturally in blood.

In embodiments, the administration is intra-parenchymal injection via stereotactic guidance into the brain of the subject. In embodiments, the neuronal injury is due to Parkinson's disease.

In embodiments, the method further comprises electrically stimulating the brain of the subject after the MSC/HSC administration, such as via deep brain stimulation (DBS).

The invention further provides pharmaceutical compositions comprising a therapeutically effective dose of substantially purified CD133+ hematopoietic stem cells (HSCs) combined with substantially purified mesenchymal stromal cells (MSCs).

The invention further provides methods for producing compositions for treating a neurological condition comprising: providing blood; isolating a substantially pure composition of CD133+ hematopoietic stem cells (HSCs) from the blood; isolating a substantially pure composition of mesenchymal stromal cells (MSCs) from the blood; and combining the composition of substantially pure CD133+ HSCs and the composition of substantially pure MSCs, to produce a composition for treating a neurological condition.

In embodiments, the HSCs and MSCs are substantially purified from blood, which may be autologous or allogeneic. In embodiments, the HSCs and MSCs are obtained from human umbilical cord blood, blood or bone marrow. In embodiments, the substantially purified compositions are isolated from at least 60%, 70%, 80%, 90% or 95% or more of the constituents found naturally in blood. In embodiments, the MSCs are further cultured from mononuclear cells in the blood (MNCs) prior to isolation.

In embodiments, the method provides for promoting tunneling nanotubules formation to promote transfer of mitochondria from HSCs and MSCs to injured neurons in the composition prior to administration.

In embodiments, the method provides for combining an effective amount of a reactive oxygen species with the composition to promote transfer of mitochondria from HSCs and MSCs to injured neurons prior to administration.

In embodiments, the method provides for activating CD73 or A2A signaling in the composition to promote transfer of mitochondria from HSCs and MSCs to injured neurons prior to administration. Type 1 IFNs, TNFa, IL-1b, prostaglandin (PG) E2, TGF-β, agonists of the wnt signaling pathway, E2F-1, CREB, Sp1, HIF1-a, Stat3, or hypoxia can be used to activate CD73 signaling.

### BRIEF DESCRIPTION OF THE DRAWINGS

FIGS. 1A-1B show HSC enhance MSC-mediated regeneration of injured dopaminergic neurons. On Day 6 of differentiation, LUHMES Dopaminergic Neurons were treated with 100 uM 6-OHDA, co-cultured for 6 hours with varying doses of UCB MSC and autoMACS-selected CD133+ HSC, fixed and stained for Tuj1 and cleaved caspase 3, an early injury marker, and visualized on an Zeiss Axiovert Z1 equipped with Apotome.2. Data represent mean+/−SD, Mann-Whitney U test.

FIGS. 2A-2B show Dual stem cells protect neurons via secreted soluble factors. On Day 6 of differentiation, LUHMES Dopaminergic Neurons were treated with 100 uM 6-OHDA, cultured for 48 h in normal differentiation media or MSC conditioned media, and stained and visualized as in FIG. 1. Data represent mean+/−SD, Mann-Whitney U test.

FIGS. 3A-3C show UCB MSC and HSC donate mitochondria to injured dopaminergic neurons. On Day 6 of differentiation, LUHMES Dopaminergic Neurons were treated with 100 uM 6-OHDA, co-cultured for 6 h with varying doses of Mitotracker Red-pre-labeled UCB MSC and autoMACS-selected CD133+ HSC, and Mitotracker transfer to neurons was monitored by fluorescence microscopy. Data represent mean+/−SD, Mann-Whitney U test.

FIGS. 4A-4C show UBC CD133+ HSC enhance neurite outgrowth, a key process in neuronal regeneration.

FIG. 5 shows that dual stem cells enhance expression of the neurite outgrowth regulator Tuj1.

FIG. 6 shows the extent to which paracrine mechanisms by themselves can contribute to regeneration.

FIG. 7 shows that HSC also transfer mitochondria to injured dopaminergic neurons via TNTs.

FIG. 8 shows that Blocking ROS reduces 6-OHDA induced injury.

FIGS. 9A-9B show CD73 mediates regeneration of injured dopaminergic neurons.

FIGS. 10A-10B show TNT formation and mitochondrial transfer from dual MSC+ HSC are actively induced by signals from injured dopaminergic neurons.

FIG. 11 shows that blocking ROS reduces 6-OHDA induced injury.

FIG. 12 shows that CD73 mediates TNT formation and mitochondrial transfer.

### DETAILED DESCRIPTION

The present invention recognizes the complexity of cell-cell interactions in regenerative therapies, that sets the basis for the embodiments of the invention for a dual stem cell therapy comprising of MSC and HSC (MSC/HSC) derived from a single UCB clinical grade graft (hence HLA and KIR identical) will have complimentary regenerative effects to control or reverse PD pathophysiology via dopaminergic neuron and supporting cells regeneration.

A second aspect of the present invention provides embodiments of the dual stem cell therapy for PD that includes injection of UCB dual stem cell graft under stereotactic guidance in subjects failing drug therapy who are undergoing placement of electrodes for deep brain stimulation (DBS). DBS has been shown to be effective for PD patients whose symptoms cannot be adequately controlled with medications. The invention provides a low dose electrical current administered over time after electrode and dual UCB MSC/HSC stem cell graft injection to provide stimulus to improve the neuro-regenerative function of the adoptively administered UCB dual stem cells.

A third aspect of the present invention provides embodiments of a stem cell therapy approach to PD treatment with the complimentary mechanisms of action of UCB derived MSC/HSC to enhance neurogenesis. The invention demonstrates transfer of mitochondria to injured dopaminergic neurons in vitro.

In embodiments, the present invention provides a novel stem cell therapeutic approach to neurological conditions, such as Parkinson's disease, based on identifying the complimentary and additive neuro-regenerative capacity of two separate populations of regenerative stem cells in UCB with potential in PD therapy comprising mesenchymal stromal cells (MSCs) and primitive CD133 expressing hematopoietic stem cells (HSCs).

In embodiments, the invention provides methods for treating injured neurons in a subject in need comprising administering to the subject a treatment effective amount of a first composition of substantially purified CD133+ HSCs and a second composition of substantially purified MSCs. In embodiments, the first and second compositions are combined prior to administration.

In embodiments, the HSCs and MSCs are substantially purified from blood, which may be autologous or allogeneic. In embodiments, the HSCs and MSCs are obtained from human umbilical cord blood, blood or bone marrow. In embodiments, the substantially purified compositions are isolated from at least 60%, 70%, 80%, 90% or 95% or more of the constituents found naturally in blood.

In embodiments, the administration is intra-parenchymal injection via stereotactic guidance into the brain of the subject. In embodiments, the neuronal injury is due to Parkinson's disease.

In embodiments, the method further comprises electrically stimulating the brain of the subject after the administration, such as via deep brain stimulation (DBS).

The invention further provides pharmaceutical compositions comprising a therapeutically effective dose of substantially purified CD133+ hematopoietic stem cells (HSCs) combined with substantially purified mesenchymal stromal cells (MSCs).

The invention further provides methods for producing compositions for treating a neurological condition comprising: providing blood; isolating a substantially pure composition of CD133+ hematopoietic stem cells (HSCs) from the blood; isolating a substantially pure composition of mesenchymal stromal cells (MSCs) from the blood; and combining the composition of substantially pure CD133+ HSCs and the composition of substantially pure MSCs, to produce a composition for treating a neurological condition. Methods and compositions are also provided for promoting mitochondrial transfer between cells, such for the treatment of PD.

In embodiments, the HSCs and MSCs are substantially purified from blood, which may be autologous or allogeneic. In embodiments, the HSCs and MSCs are obtained from human umbilical cord blood, blood or bone marrow. In embodiments, the substantially purified compositions are isolated from at least 60%, 70%, 80%, 90% or 95% or more of the constituents found naturally in blood. In embodiments, the MSCs are further cultured from mononuclear cells in the blood (MNCs) prior to isolation.

In certain embodiments, the methods and manufacturing steps comprise. a simple novel technique that ensures robust UCB MSC outgrowth after CD133 HSC selection, thereby allowing dual stem cell therapeutic cell populations from a single UCB clinical grade graft at cell doses suitable for human PD therapy.

The invention further provides for promoting tunneling nanotubules formation to promote transfer of mitochondria from HSCs and MSCs to injured neurons in the composition prior to administration. The invention provides that combining an effective amount of a reactive oxygen species with the composition promotes transfer of mitochondria from HSCs and MSCs to injured neurons prior to administration.

The invention further provides that promoting CD73 or A2A signaling pathways in the composition promotes transfer of mitochondria from HSCs and MSCs to injured neurons. Type 1 IFNs, TNFa, IL-1b, prostaglandin (PG) E2, TGF-β, agonists of the wnt signaling pathway, E2F-1, CREB, Sp1, HIF1-a, Stat3, or hypoxia can be used to promote CD73 signaling.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, “patient” or “subject” means an animal subject to be treated, with human patients being preferred.

As used herein, “proliferation” or “expansion” refers to the ability of a cell or population of cells to increase in number.

As used herein, the term “MSC” embraces mesenchymal stromal cells, which can be derived from mononuclear cells. Techniques for obtaining MSCs are well-known in the art and are further described in U.S. Provisional Patent Application No. 62/684,854, which is incorporated herein by reference.

As used herein, the term “HSC” embraces hematopoietic stem cells that express the CD133+ phenotype.

As used herein, “substantially purified” or “substantially pure” refers to the characteristic of a population of first substances being removed from the proximity of a population of second substances, such as those with which the first substances are found in nature, wherein the population of first substances is not necessarily devoid of the second substance, and the population of second substances is not necessarily devoid of the first substance. However, a population of first substances that is “substantially purified” from a population of second substances has a measurably lower content of second substances as compared to the non-separated mixture of first and second substances. In one aspect, at least 30%, 50%, 60%, 70%, 80%, 90%, 95%, 98%, 99%, or more of the second substance is removed from the first substance.

As used herein, “therapeutically effective” refers to an amount of cells that is sufficient to treat or ameliorate, or in some manner reduce the symptoms associated with a neuronal injury. When used with reference to a method, the method is sufficiently effective to treat or ameliorate, or in some manner reduce the symptoms associated with an aberrant response. For example, an effective amount in reference to a disease is that amount which is sufficient to block or prevent its onset; or if disease pathology has begun, to palliate, ameliorate, stabilize, reverse or slow progression of the disease, or otherwise reduce pathological consequences of the disease. In any case, an effective amount may be given in single or divided doses.

As used herein, the term “treatment” embraces at least an amelioration of the symptoms associated with the aberrant condition in the patient, where amelioration is used in a broad sense to refer to at least a reduction in the magnitude of a parameter, e.g. a symptom associated with the condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

In some embodiments, MSCs and CD133+ HSCs can be obtained from blood, including umbilical cord blood, originating from a variety of animal sources including, for example, humans. Thus, some embodiments include providing human umbilical cord blood from a single allogeneic donor as the source for cells used in the present invention.

In some embodiments, naïve CD133+ cells are substantially separated from other cells in umbilical cord blood to form a purified CD133+ HSC cell composition. Methods for separating/purifying CD133+ HSCs from blood are well-known in the art, and further described in the Examples below. Techniques include Ficoll-Paque density gradient separation to isolate viable mononuclear cells from blood using a simple centrifugation procedure, and affinity separation to separate CD133+ cells from the mononuclear cells. Exemplary affinity separation techniques can include, for example, magnetic separation (e.g. antibody-coated magnetic beads) and fluorescence-activated cell sorting. The substantially purified CD133+ HSCs can be further expanded in culture. In some embodiments, at least 75%, 85%, 86%, 87%, 88%, 89%, 90%, 91%, 92%, 93%, 94%, 95%, 96%, 97%, 98%, 99%, or more of the cells of the resulting composition are CD133+ HSCs. In some embodiments, the purity of CD133+ HSCs is equal to or greater than 75%, 85%, 86%, 87%, 88%, 89%, 90%, 91%, 92%, 93%, 94%, 95%, 96%, 97%, 98%, 99%, or more.

In some embodiments, MSCs are substantially purified from other cells and materials in the remaining umbilical cord blood. Methods for separating/purifying MSCs are well-known in the art, and further described in the Examples below. Techniques can include affinity separation methods such as magnetic cell sorting (e.g. antibody-coated magnetic beads) and fluorescence-activated cell sorting to separate cells from other cells. In one non-limiting example, cells are purified using magnetic separation kits. The substantially purified MSCs can be further expanded in culture. In some embodiments, at least 75%, 85%, 86%, 87%, 88%, 89%, 90%, 91%, 92%, 93%, 94%, 95%, 96%, 97%, 98%, 99%, or more of the cells of the substantially purified compositions of cells. In some embodiments, the purity of cells is equal to or greater than 75%, 85%, 86%, 87%, 88%, 89%, 90%, 91%, 92%, 93%, 94%, 95%, 96%, 97%, 98%, 99%, or more. In some embodiments, at least 75%, 85%, 86%, 87%, 88%, 89%, 90%, 91%, 92%, 93%, 94%, 95%, 96%, 97%, 98%, 99%, or more of the cells are substantially purified.

In some embodiments, the remaining cells in blood from which HSCs have been removed are cultured to form an expanded mononuclear cell composition. Thus, in embodiments, the cultured mononuclear cell composition is expanded to produce a larger population of MSCs. The expansion step can use culture techniques and conditions well known in the art. In certain embodiments, the cells are expanded by maintaining the cells in culture for about 1 day to about 3 months. In further embodiments, the cells are expanded in culture for about 2 days to about 2 months, for about 4 days to about 1 month, for about 5 days to about 20 days, for about 6 days to about 15 days, for about 7 days to about 10 days, and for about 8 days to about 9 days. The mesenchymal stromal cells (MSC) can be derived from any suitable source (e.g. bone marrow, adipose tissue, placental tissue, umbilical cord blood, umbilical cord tissue).

In some embodiments, the cultured cells are expanded at least 2-fold, at least 3-fold, 4, 5, 6, 7, 8, 9, 10, 50, 100, 200, 300, 500, or at least 800-fold. In some embodiments, compositions comprising the expanded cells contain a clinically relevant number or population of cells. In some embodiments, compositions include about 10¹, about 10⁴, about 10⁵ cells, about 10⁶ cells, about 10⁷ cells, about 10⁸ cells, about 10⁹ cells, about 10¹⁰ cells or more. In some embodiments, the number of cells present in the composition will depend upon the ultimate use for which the composition is intended, e.g., the disease or state or condition, patient condition (e.g., size, weight, health, etc.), and other health-related parameters that a skilled artisan would readily understand. In addition, in some embodiments, the clinically relevant number of cells can be apportioned into multiple infusions that cumulatively equal or exceed the desired administration, e.g., 10⁹ or 10¹⁰ cells.

The substantially purified cells can be used immediately. The substantially purified cells can also be frozen at liquid nitrogen temperatures and stored for long periods of time, being thawed and capable of being used. The cells may be stored, for example, in DMSO and/or FCS, in combination with medium, glucose, etc.

In some embodiments, a therapeutically effective amount of a composition comprising umbilical cord blood derived MSC and HSC can be administered to the subject with a pharmaceutically acceptable carrier. Administration routes may include any suitable means, including, but not limited to, intra-parenchymal injection via stereotactic guidance directly into the diseased target tissue (e.g., brain), or intravascularly (intravenously or intra-arterially). In some embodiments, the particular mode of administration selected will depend upon the particular treatment, disease state or condition of the patient, the nature or administration route of other drugs or therapeutics administered to the subject, etc.

In some embodiments, about 10⁵-10¹¹ cells can be administered in a volume of a 5 ml to 1 liter, 50 ml to 250 ml, 50 ml to 150, and typically 100 ml. In some embodiments, the volume will depend upon the disorder treated, the route of administration, the patient's condition, disease state, etc. The cells can be administered in a single dose or in several doses over selected time intervals, e.g., to titrate the dose.

In one aspect, the compositions and methods disclosed herein are directed to modulating an aberrant neurological condition in a subject, such as Parkinson's disease, by administering the umbilical cord blood derived mesenchymal stromal cells and hematopoietic stem cells disclosed herein.

The umbilical cord blood derived cells including mesenchymal stromal cells and hematopoietic stem cells disclosed herein can be used to treat, alleviate or ameliorate the symptoms of or suppress a wide variety of neurological disorders. In some embodiments, the neurological disorders include, but are not limited to, Parkinson's disease, amyotrophic lateral sclerosis (ALS), Primary lateral sclerosis, muscular atrophy, Progressive bulbar palsy, Huntington's disease, Friedreich's Ataxia and Alzheimer's disease.

The methods find use in the treatment of a variety of different conditions and situations in which the response in a patient is desired. By way of example, but not by way of limitation, a composition comprising HSCs and MSCs as disclosed herein may be administered during a related surgery, such as a procedure for installation of electrodes for deep brain stimulation of cells near the substantia nigra, a region of the midbrain affected by PD. As discussed above, The Food and Drug Administration approved DBS as a treatment for essential tremor and Parkinson's disease (PD) in 1997. This surgery places microelectrodes for deep brain stimulation and implants a medical device called a neurostimulator, which sends electrical impulses to specific parts of the brain. Typically, a thin lead with multiple electrodes is implanted in the globus pallidus, nucleus ventralis intermedius thalami, or subthalamic nucleus, and electric pulses are used therapeutically. The lead from the implant is extended to the neurostimulator under the skin in the chest area. DBS effects the physiology of brain cells and neurotransmitters by sending high-frequency electrical impulses into specific areas of the brain, which can mitigate symptoms and directly diminish the side effects induced by PD medications, allowing a decrease in medications, or making a medication regimen more tolerable.

In some embodiments, it is convenient to administer the cells in a pharmaceutically acceptable carrier, such as an artificial gel, or in clotted plasma, or by utilizing other controlled release mechanism known in the art. In embodiments, the invention can be provided in two frozen components, which are thawed, combined and applied to the injured neural tissue. In embodiments, the combined HSCs and MSCs gels or solidifies within 1-5 minutes, or 1-2 minutes of application in vivo within the injured tissue.

Caspase 3 is a quantitative measure of dopaminergic neuronal injury and regeneration. Specifically, it is an early indicator of injury at a stage that can be rescued from cell death. Therefore, therapeutics that reduce caspase 3 cleavage are strong candidates to promote neuronal regeneration. This invention provides for measuring cleaved caspase 3 expression to quantitatively asses therapeutic efficacy of disclosed therapies.

Neurite outgrowth after injury is a quantitative measure of dopaminergic neuronal regeneration. Neurites are extensions from the neuronal cell body that are critical for transmission of signals between cells including dopamine. In addition to caspase 3 cleavage, neurite damage is a hallmark of neuronal injury by 6-Hydroxydopamine (6-OHDA), hypoxia, and other insults. Therefore, therapeutics that induce neurite outgrowth are promising candidates for neuronal regeneration.

6-OHDA is a toxic metabolite that induces hallmarks of dopaminergic neuronal injury including mitochondrial dysfunction and neurite damage. Thus, 6-OHDA dopaminergic neural injury model allows quantitative assessment of stem cell therapeutic efficacy. Furthermore, a hypoxia induced injury model can also be used to assess therapeutic efficacy. Specifically, incubating neurons in a gas-controlled hypoxia chamber at low oxygen induces dopaminergic neuronal injury that can be measured via injury markers such as cleaved caspase 3 and neurite outgrowth. Using these injury models the invention has shown the enhanced therapeutic efficacy of a dual cell MSC+ HSC therapeutic.

The invention provides that concurrent administration of MSC and HSC significantly reverses neuronal apoptosis and enhances neurite formation to a surprisingly greater extent than either stem cell population alone at equivalent cell doses.

MSC and HSC-mediated restoration of dopaminergic function is mediated by mechanisms including but not limited to: 1) additive neurotrophic and vasculogenic effects of MSC and HSC via paracrine mechanism, 2) direct cell-cell interactions including transfer of mitochondria to injured dopaminergic neurons. Prior work has identified UCB-MSC-derived factors including thrombospondin that play a critical role in neuroprotection.⁴³ UCB MSC conditioned media is sufficient to rescue dopaminergic neurons from 6-OHDA induced injury, supporting a paracrine mechanism of action.

With respect to direct cell-cell interactions mediating neuro-regenerative function, a number of studies indicate that mitochondrial dysfunction plays an important role in PD pathogenesis; indeed, several reports suggest effective neuronal rescue depends on direct cell-cell contact that promote donation of mitochondria from therapeutic stem cells to injured neurons.⁴⁴⁻⁵⁴ The invention shows that both UCB MSC and HSC each form close associations with—, and donate mitochondria to—, injured dopaminergic neurons, further suggesting their potential additive benefits in UCB MSC/HSC neuro-regeneration. The invention shows that mitochondrial transfer occurs via tunneling nanotubules (TNT). TNT had not previously been observed in HSCs. The invention further identifies reactive oxygen species (ROS) as a major mechanism driving TNT formation and mitochondrial transfer and that CD73 signaling mediates TNT formation, mitochondrial transfer, and neuronal regeneration. The invention also shows that compared with paracrine effects, cell-cell interactions provide for a greater role in the regeneration process.

TNT formation and mitochondrial transfer can be induced by compounds such as M-Sec, also known as tumor necrosis factor-α-induced protein, actin polymerization factors including the Rho GTPases family Rac1 and Cdc42, and their downstream effectors WAVE and WASP, and by the expression of the leukocyte specific transcript 1 (LST1) protein in HeLa and HEK cell lines, as described in DuPont et al., Front. Immunol., 25 Jan. 2018 (https://doi.org/10.3389/fimmu.2018.00043). TNT and mitochondrial transfer can also be induced by compounds such as doxorubicin and other anthracycline analogs and other agents that cause cellular stress responses, as described in Desir et al, Scientific Reports, volume 8, Article number: 9484 (2018). TNT and mitochondrial transfer can be inhibited by Cytochalasin B, and nucleoside analogs, such as cytarabine (cytosine arabinoside, AraC), as described in Omsland et al., Scientific Reports, volume 8, Article number: 11118 (2018). Furthermore, Cytochalasin D is cell permeable and an actin inhibitor. Cytocalasin D can cause significant reduction in TNT formation, as shown in Sienz-de-Santa-Marfa et al., Oncotarget, 2017. See also Hanna et al. Scientific Reports (2017); Keller et al. Invest Ophthalmol Vis Sci. (2017).

Treg express apyrases (CD39) and ecto-5′-nucleotidase (CD73) that promote mitochondrial transfer. CD39/CD73 may be upregulated by using type 1 IFNs, TNFa, IL-1b, prostaglandin (PG) E2, TGF-β, agonists of the wnt signaling pathway, E2F-1, CREB, Sp1, HIF1-a, Stat3, and hypoxia. See Beavis et al., Trends in Immunology (2012); Bao et al., Int'l J. of Molecular Med (2012); Regateiro et al., Eur. J. Immunol (2011); Synnestvedt et al., J. Clin Invest (2002); Eltzschig et al., J of Exp. Med (2003); Eltzschig et al., Blood (2009); and Chalmin et al., Immunity (2012). Gfi-1 represses CD39/CD73 expression, as described in Chalmin, Immunity (2012). CD39/CD73 may also be inhibited using blocking antibodies or pharmacological inhibitors such as POM1 (a E-NTPDases inhibitor), and Adenosine 5′-(α,β-methylene)diphosphate.

The invention provides that HSC and MSC exert complimentary neuro-regenerative effects via secretion of pro-vasculogenic and neurotrophic factors, as well as mitochondrial transfer. The invention also identifies the novel role of HSCs in promoting neurite growth and enhancing levels of neurite growth regulator Tuj1.

### EXAMPLES

### Example 1—MSC and HSC Purification from UBS and Ex Vivo Expansion

The purpose of this Example is to describe an exemplary procedure for isolating mononuclear cells from umbilical cord blood and for isolating potential mesenchymal stromal cells that might have become attached to the inner surfaces of the cord blood collection bag, and to isolate CD133+ hematopoietic stem cells from the same unit.

Make sure cord blood tubing is inside of a 50 mL falcon tube, lift bag at an appropriate angle so blood flows naturally into tube. Drain 50 mL into each tube. Dilute 25 mL of blood with 25 mL of PBS (1:1 blood to PBS dilution). After dilution, turn tubes upside down 3-4 times to mix together. Pipet 15 mL of Ficoll-Paque density gradient medium into a 50 mL Sepmate Tube. Place the pipet tip against the vertex near the bottom of the tube. Slowly fill bottom of tube, make sure no bubbles are present. Very slowly pipet 25 mL of the diluted blood-PBS mixture into the 50 mL Sepmate tube that contains the Ficoll. Place the pipette tip against the side of the tube and slowly release the mixture into the tube, making sure that no blood enters the bottom portion of the sepmate tube. Keep Sepmate tube vertical.

Centrifuge (at 2400 rpm (1200×g) for 40 minutes with the brake on (Acceleration program 9 and deceleration Program 6) [ThermoFisher Sorvall Legend XTR]. After centrifugation (RBCs remain on the bottom) the top layer is pipetted off. The top layer contains the buffy coat, along with the plasma. Combine this fraction with layers from other tubes into a 50 mL falcon tube. Centrifuge these tubes at 1200 rpm for 8 minutes. Aspirate liquid leaving the pellet at the bottom of the tube.

Wash MNCs with 1×PBS (add up to 50 mL) and spin down at 1200 rpm for 8 minutes. Aspirate liquid and wash MNCs again with 1×PBS and spun down at 1200 rpm for 8 minutes. Depending on nucleated red blood cell concentration, ACK lysing buffer can be used to lyse RBC. Pipette 5 mL of buffer to suspend the pellet. After 5 minutes, spin down the tubes at 1200 rpm for 8 minutes. Wash cells with 1×PBS, split into two tubes 1 and 2 and spin down at 1200 rpm for 8 minutes. Suspend cell pellet 1 with 10 mL of IMDM with 20% HS and cell pellet 2 with 1 mL of MACS buffer and count cells. Take 10 μl of the cell suspensions and add it to an Eppendorf. Add 90 μl of IMDM to the tube. Finally, add 100 μl of Trypan Blue stain to the same tube. All 10 μl of this mixture to hemocytometer and count cells.

For cell suspension 1, follow steps below. For cell suspension 2, stain the cell suspension using CD133 Microbeads (Miltenyi) according to manufacturer's instructions.

For cell suspension 1, plate in 25 cm² flask with cell density being 1×10⁶ cells/cm². There will be two flasks A and B. A will only contain these MNCs obtained through this procedure. B will contain MNCs along with cells obtained from the cord blood bag. This process will be detailed further below. Cells labeled A are plated in IMDM with 20% HS with appropriate volume of IMDM to maintain cell density 1×10⁶ cells/cm² in an appropriate size flask. Cells are placed in a CO₂ incubator at 37° C. and 5% CO₂.

After blood is drained from the cord blood bag, add 150 mL of Accutase Dissociation Buffer to the cord blood bag with a 30 mL syringe. Place the tip of the syringe inside of the cord blood unit tubing and pour fluid in. Accutase should not be heated at 37° C., thaw in fridge overnight. After 150 mL of Accutase buffer has been added, wrap Parafilm around the end of the tubing of the cord blood unit (to ensure that no liquid leaves the bag). Place cord blood bag into a sterile transport bin and then place on a shaker 1000 rpm for 10 minutes. This process is done at room temperature. Drain cord blood bag into 50 mL Falcon tubes. The Cord blood bag tubing is placed into the 50 mL Falcon tube and the bag is again lifted until the Falcon tube is filled. Repeat infusion of Accutase Dissociation Buffer into the cord blood bag, shaking for 10 min and draining. Follow with two PBS rinses (100 mL) of the cord blood bag to ensure collection of all cells.

Spin down both the Falcon tubes containing the PBS rinse and also Accutase Dissociation Buffer at 1200 rpm for 8 minutes. Aspirate liquid and combine cells into a single 50 mL Falcon tube. Wash these cells in PBS and spin down at 1200 rpm for 8 minutes. Suspend the cell pellet in Complete IMDM media (IMDM, 20% HS, 100 U/ml Pen/Strep, 2 mM Glutamine) supplemented with fresh 40 ng/ml bFGF. Add these cells to the MNC Cells Labeled B from above. Plate these cells in Complete IMDM+FGF in an appropriate sized flask and concentration to maintain 1×10⁶ cells/cm2 with depth. Add media to the flask after 72-120 hours. Replace media after 120-168 hours. Examine cells under a light microscope frequently.

For MSC culture and cryopreservation, aspirate medium from flask. Treat with 5 mL TrypLE Select for 3 minutes in the incubator. Rinse the flask to collect cells and spin down at 1200 rpm for 5 minutes. Aspirate liquid. Resuspend cell pellet in Complete IMDM media with 20% HS for cell culture or prepare cells for cryopreservation in 90% FBS, 10% DMSO. Cells should not be passaged more than ˜5 times as they begin to lose MSC phenotypic characteristics.

For CD133+ HSC isolation, adjust cell density to 108 cells/mL. Add 1:11 dilution of FcBlock and 1:11 CD133 Microbeads. Incubate in the fridge (not on ice) for 30 minutes. Wash cells with 5 mL of MACS buffer and spin down at 1200 rpm for 10 minutes. Aspirate liquid. Resuspend cell pellet in MACS buffer and perform CD133 isolation using AutoMACS Posselds program for rare cell types, collecting both CD133 positive fraction (pos1) and CD133 negative cells (neg). Spin down isolated cells at 1200 rpm for 10 minutes. Aspirate liquid. For use of fresh HSCs in experiments, resuspend cell pellet in Complete IMDM media (without FGF) and culture up to ˜1-2 days prior to use in experiment. For cryopreservation, resuspend cells in 90% FBS, 10% DMSO and freeze 10⁶ cells/vial.

For CD133+ HSC expansion, CD133+ HSCs isolated by AutoMACS Posselds program in the positive fraction are expanded by plating them at between 250,000 to 1,000,000 cells per well of 24 well plate in 1 mL of culture media supplemented with 10 ng/ml each of TPO, FLT3, and SCF. Every 2-3 days, cells are pipetted up and down to detach any weakly adherent cells and subcultured 1:5 ratio, for 10 days. On day 10 of ex vivo expansion, cells are re-selected for CD133+ cells (Following SOP CD133 HSC Isolation). Following CD133 cell isolation, remove an aliquot for characterization (SOP Characterization of CD133+ HSC Purity by Flow Cytometry). Remaining cells are cryopreserved in 90% FBS, 10% DMSO and freeze 106 cells/vial.

The expected results are that 100-200 Million Mononuclear cells will be obtained per 100 mL of cord blood. 60-80% Cord Blood Units processed to include bag wash cells will generate Mesenchymal Stromal Cells. 40% of cord blood MNCs alone will generate MSC. These MSCs are grown to confluency, passaged, and analyzed via FACS, and MLR suppression assay. Cell preps that contain cells obtained from the cord blood bag that are added to the MNC fraction will be more likely to generate MSCs than cell preps containing the MNC fraction that was plated by itself. 0.5-1 million CD133+ HSCs will be obtained per 100 mL of cord blood. 55×10⁶ cells were obtained (55-fold expansion of initial 106 CD133-selected HSCs) after 10 days of ex vivo expansion. 15% of the 55×10⁶ expanded cells express CD133; thus, the total CD133 HSC yield is ˜10×10⁶ cells.

### Example 2—Novel Role for HSCs in Promoting Neurite Outgrowth

Neurites are extensions from the neuronal cell body that play a key role in the normal function of neurons and connectivity and communication between neurons. A hallmark of neuronal injury by hypoxia or environmental toxins such as 6-OHDA is damage to neurites, manifested as consolidation of neurite networks and degradation of neurites. Neurite outgrowth is a key process that contributes to regeneration and can be quantified by microscopic analysis. MSCs secrete neuronal growth promoting factors, however MSCs have only shown modest benefits for neurite outgrowth. The effects of HSCs on neurite outgrowth has not been studied, however HSCs secrete proteins including SDF-1, which have been shown to promote neurite outgrowth. This invention discloses that HSCs can enhance neurite outgrowth after injury.

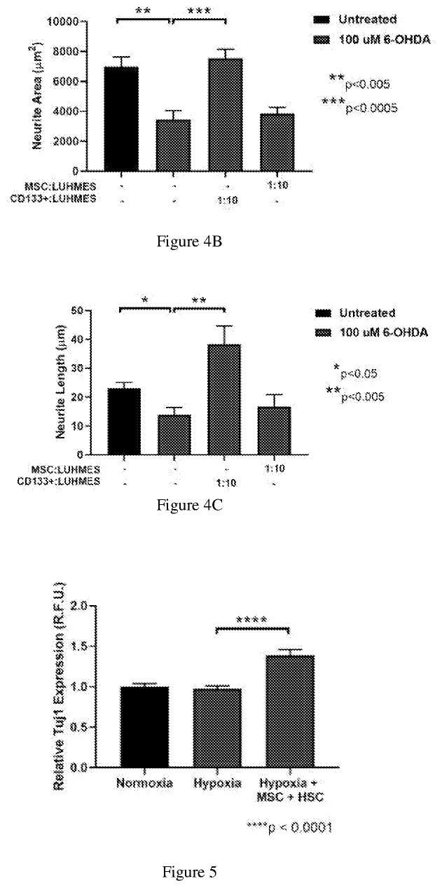

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 35 mm #1.5 glass bottom dishes (Cellvis Catalog #D35-20-1.5-N) in fresh differentiation media. On Day 6 of differentiation, cells were treated with 100 uM 6-OHDA for 45 minutes, washed and co-cultured with UCB MSC and/or CD133+ HSC at 10:1:LUHMES:MSC+ HSC ratio and incubated for 72 hours. After 3 days, cells were then processed for caspase 3/Tuj1 co-staining. Cells were fixed in 2% paraformaldehyde for 15 minutes at room temperature. Cells were washed in PBS, stained overnight at 4 degrees C. with rabbit Cleaved Caspase 3 antibody (Cell Signaling Technologies, 1:400) and mouse Tuj1 antibody (Biolegend, 1:400) in 0.1% saponin, 0.1% BSA/PBS pH 7.4. Cells were washed in PBS and stained for 1 hour at room temperature with Alexa Fluor 488 anti-mouse IgG and Alexa Fluor 647 anti-rabbit IgG (1:250, Invitrogen). Cells were washed in PBS and imaged on a Zeiss Axiovert Fluorescence Microscope, Z-stack images were collected on a Zeiss Axiovert Fluorescence Microscope, 60× oil immersion objective equipped with Apotome.2. FIG. 4A represents 3D reconstructions of z-stack images and data are representative of 3 independent experiments. Statistical significance was determined by Mann-Whitney U-test using Graphpad Prism 8.0 software.

Neurite paths were traced and neurite area was measured using the Neurolucida 360 neurite analysis software. The area of each neurite was measured in each image field, and data represent the average, total or maximum neurite area from at least four image fields. The data in FIGS. 4B-4C represent the mean+/−standard deviation from one representative experiment of 3 independent experiments. Statistical significance was determined by unpaired t-test using Graphpad Prism 8.0 software.

Injury by neurotoxin treatment reduces overall neurite area, and HSCs significantly enhance neurite outgrowth after toxin-induced injury, as shown by complete recovery of neurite area and length to levels observed in uninjured neurons. Although MSCs do aid recovery of neurites in some cases, overall HSCs have a significantly greater regenerative benefit for neurite outgrowth after injury.

### Example 3—Dual MSC and HSC Cell Therapy

This Example provides allogeneic UCB derived dual stem cells (MSC/CD133+ HSC) biologic therapy with provision of optimal HLA match for each patient based on global UCB inventory search of clinical grade UCB networked via the National Marrow Donor Program.

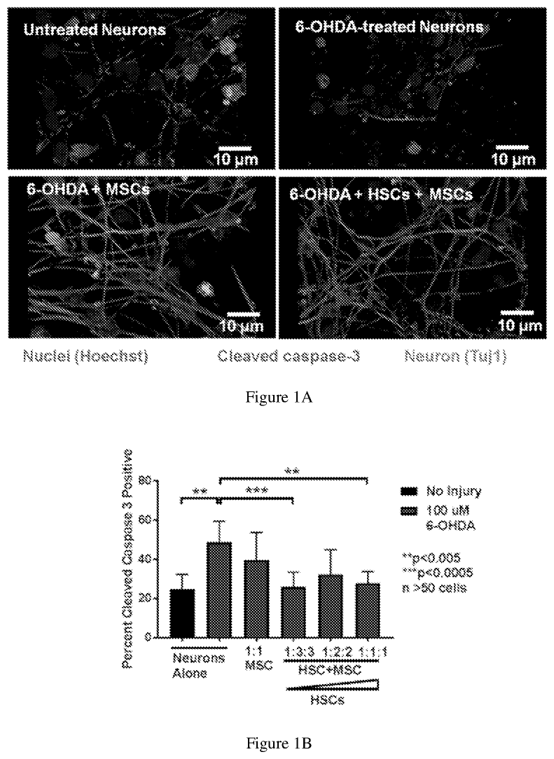

FIGS. 1A-1B show that HSC enhance MSC-mediated regeneration of injured dopaminergic neurons. On Day 6 of differentiation, LUHMES Dopaminergic Neurons were treated with 100 uM 6-OHDA, co-cultured for 6 hours with varying doses of UCB MSC and autoMACS-selected CD133+ HSC, fixed and stained for Tuj1 and cleaved caspase 3, an early injury marker, and visualized on an Zeiss Axiovert Z1 equipped with Apotome.2. Data represent mean+/−SD, Mann-Whitney U test.

MSC and HSC isolation and culture: Human UCB was collected into collection bags containing citrate dextrose (Allegiance, Deerfield, Ill.). Mononuclear cells were isolated by Ficoll-Paque PLUS (GE Healthcare Life Sciences, Piscataway, N.J.) density gradient centrifugation with SepMate-50 tubes (STEMCELL Technologies). For HSC isolation, MNCs were labeled with CD133 microbeads (Miltenyi) per manufacturer's protocol. The labeled CD133+ HSCs were isolated on an AutoMACS system (Miltenyi) using the Posselds program. Yield was routinely 10⁶ cells per UCB unit and purity was routinely assessed by CD133 staining by flow cytometry to be >90%. HSCs were cryopreserved until use in experiments. MSCs were generated by culturing MNCs in IMDM media containing 20% human serum (Abnova), 100 U/ml Penicillin/Streptomycin, 2 mM glutamine, and supplemented fresh with 40 ng/ml basic FGF (Sigma). Media was replaced every 2-3 days and subcultured in T75 tissue culture flasks pre-coated with 10 ug/ml fibronectin (Sigma). After 3-4 weeks of culture, cells were cryopreserved and stored until use in experiments. UCB MSCs and HSCs were thawed and rested 1-2 days prior to use in experiments.

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 35 mm #1.5 coverglass (Cellvis) in fresh differentiation media. On Day 6 of differentiation, cells were treated with 100 uM 6-OHDA for indicated time periods, washed once and indicated combinations of UCB stem cells were added in direct co-culture with neurons. After 6 hours, cells were fixed in 2% paraformaldehyde for 15 minutes at room temperature. Cells were washed in PBS, stained overnight at 4 degrees C. with rabbit Cleaved Caspase 3 antibody (Cell Signaling Technologies, 1:400) and mouse Tuj1 antibody (Biolegend, 1:400) in 0.1% saponin, 0.1% BSA/PBS pH 7.4. Cells were washed in PBS and stained for 1 hour at room temperature with Alexa Fluor 488 anti-mouse IgG and Alexa Fluor 647 anti-rabbit IgG (1:250, Invitrogen). Cells were washed in PBS and imaged on a Zeiss Axiovert Fluorescence Microscope, 40× objective. FIG. 1A represents 3D reconstructions of z-stacks acquired with the Apotome.2 for structured illumination. The percent cleaved caspase 3 positive Tuj1+ neurons was determined by manual counting from fluorescence images. Data represent the mean+/−standard deviation from analysis of n>50 cells from at least 4 image fields and are representative of 3 independent experiments.

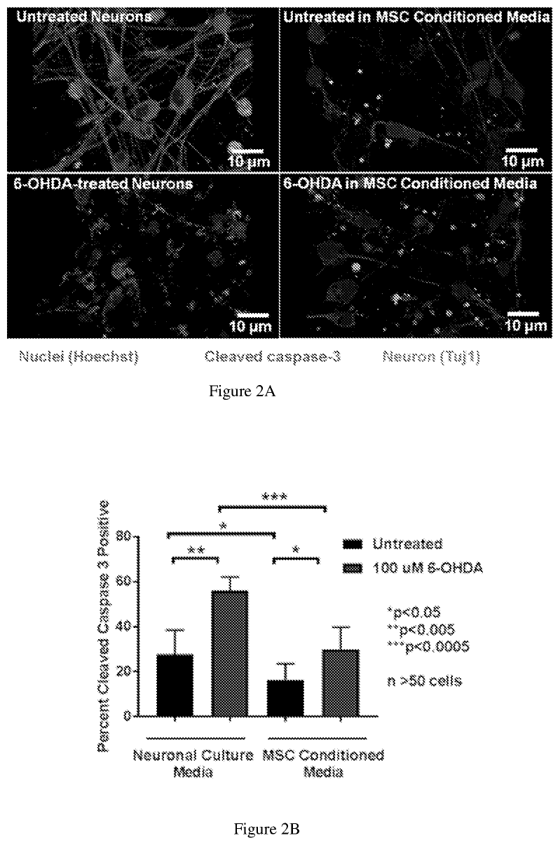

FIGS. 2A-2B show that dual stem cells (CD133+ HSCs and MSCs) protect neurons via secreted soluble factors. On Day 6 of differentiation, LUHMES Dopaminergic Neurons were treated with 100 uM 6-OHDA, cultured for 48 h in normal differentiation media or MSC conditioned media, and stained and visualized as in FIG. 1A. Data represent mean+/−SD, Mann-Whitney U test.

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 35 mm #1.5 coverglass (Cellvis) in fresh differentiation media. On Day 6 of differentiation, cells were treated with 100 uM 6-OHDA for 45 minutes, washed and incubated for 3 days in indicated media (differentiation media or MSC conditioned media collected from 5 day cultured UCB MSCs). Cells were then fixed in 2% paraformaldehyde for 15 minutes at room temperature. Cells were washed in PBS, stained overnight at 4 degrees C. with rabbit Cleaved Caspase 3 antibody (Cell Signaling Technologies, 1:400) and mouse Tuj1 antibody (Biolegend, 1:400) in 0.1% saponin, 0.1% BSA/PBS pH 7.4. Cells were washed in PBS and stained for 1 hour at room temperature with Alexa Fluor 488 anti-mouse IgG and Alexa Fluor 647 anti-rabbit IgG (1:250, Invitrogen). Cells were washed in PBS and imaged on a Zeiss Axiovert Fluorescence Microscope, 40× objective. FIG. 2A represents 3D reconstructions of z-stacks acquired with the Apotome.2 for structured illumination. The percent cleaved caspase 3 positive Tuj1+ neurons was determined by manual counting from fluorescence images. Data represent the mean+/−standard deviation from analysis of n>30 cells from at least 4 image fields and data from two independent experiments are shown.

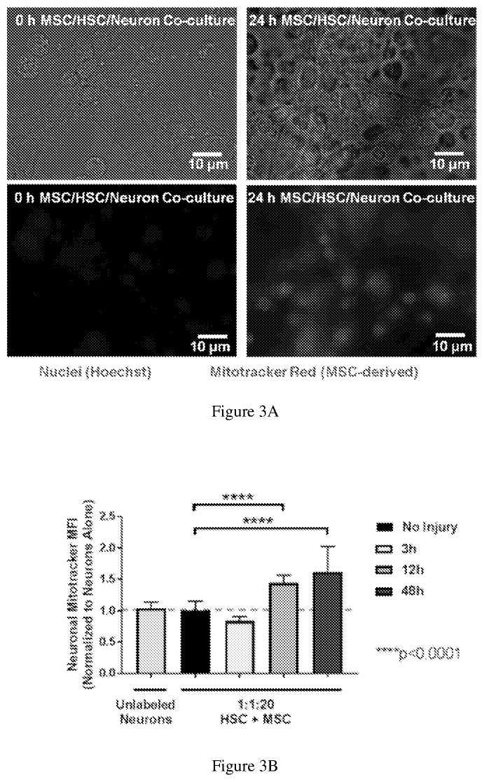

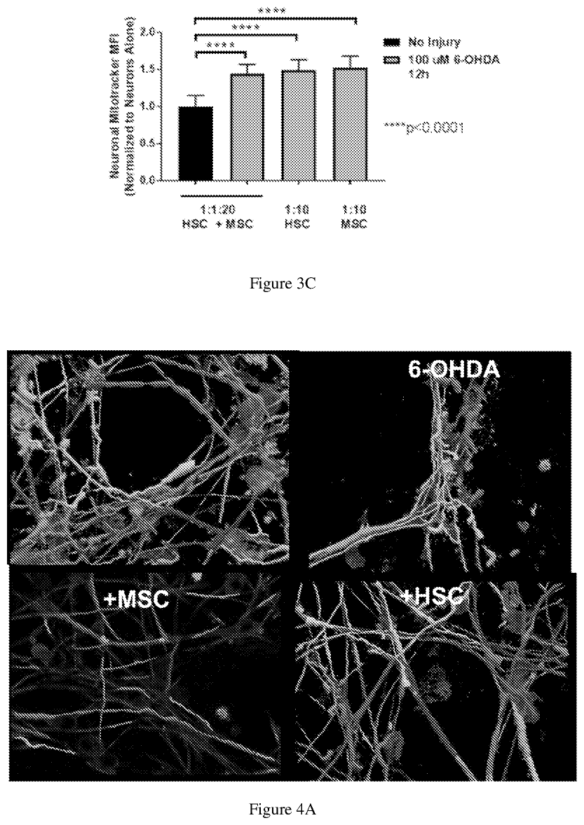

FIGS. 3A-3C show that UCB MSC and HSC donate mitochondria to injured dopaminergic neurons. On Day 6 of differentiation, LUHMES Dopaminergic Neurons were treated with 100 uM 6-OHDA, co-cultured for 6 h with varying doses of Mitotracker Red-pre-labeled UCB MSC and autoMACS-selected CD133+ HSC, and Mitotracker transfer to neurons was monitored by fluorescence microscopy. Data represent mean+/−SD, Mann-Whitney U test.

MSC and HSC isolation and culture: Human UCB was collected into collection bags containing citrate dextrose (Allegiance, Deerfield, Ill.). Mononuclear cells were isolated by Ficoll-Paque PLUS (GE Healthcare Life Sciences, Piscataway, N.J.) density gradient centrifugation with SepMate-50 tubes (STEMCELL Technologies). For HSC isolation, MNCs were labeled with CD133 microbeads (Miltenyi) per manufacturer's protocol. The labeled CD133+ HSCs were isolated on an AutoMACS system (Miltenyi) using the Posselds program. Yield was routinely 10⁶ cells per UCB unit and purity was routinely assessed by CD133 staining by flow cytometry to be >90%. HSCs were cryopreserved until use in experiments. MSCs were generated by culturing MNCs in IMDM media containing 20% human serum (Abnova), 100 U/ml Penicillin/Streptomycin, 2 mM glutamine, and supplemented fresh with 40 ng/ml basic FGF (Sigma). Media was replaced every 2-3 days and subcultured in T75 tissue culture flasks pre-coated with 10 ug/ml fibronectin (Sigma). After 3-4 weeks of culture, cells were cryopreserved and stored until use in experiments. UCB MSCs and HSCs were thawed and rested 1-2 days prior to use in experiments. Just prior to co-culture with neurons, MSCs and HSCs were labeled with 50 nM Mitotracker Red for 30 minutes at 37 degrees C. Cells were washed and co-cultured with neurons.

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 35 mm #1.5 coverglass (Cellvis) in fresh differentiation media. On Day 6 of differentiation, cells were treated with 100 uM 6-OHDA for indicated time periods, washed once and indicated combinations of Mitotracker Red-labeled UCB stem cells were added in direct co-culture with neurons. After 0 or 24 hours, cells were imaged on a Zeiss Axiovert Fluorescence Microscope, 40× objective. Data represent the mean fluorescence intensity of Mitotracker Red staining in neurons quantified in ImageJ and normalized to background staining present in unstained neurons.

### Example 4—Dual Stem Cells Enhance Expression of the Neurite Outgrowth Regulator Tuj1

FIG. 5 shows that dual stem cells enhance expression of the neurite outgrowth regulator Tuj1. LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 24 well plates in fresh differentiation media. On Day 6 of differentiation, 24 well plates are placed in a hypoxia chamber (C-Chamber, Biospherix) and cells are exposed to 5% oxygen for 24 hours delivered using a custom gas mixture consisting of 5.25% carbon dioxide and 94.75% Nitrogen delivered using an oxygen controller (P15 O2 controller, Biospherix). After 24 hours, plates are removed from hypoxia chamber and co-cultured with PKH26-labeled UCB MSC and/or UCB CD133+ or CD133− HSC in indicated ratio and incubated for 72 hours.

After 3 days, cells were then processed for caspase 3/Tuj1 co-staining. Cells were fixed in 2% paraformaldehyde for 15 minutes at room temperature. Cells were washed in PBS, stained overnight at 4 degrees C. with rabbit Cleaved Caspase 3 antibody (Cell Signaling Technologies, 1:400) and mouse Tuj1 antibody (Biolegend, 1:400) in 0.1% saponin, 0.1% BSA/PBS pH 7.4. Cells were washed in PBS and stained for 1 hour at room temperature with Alexa Fluor 488 anti-mouse IgG and Alexa Fluor 647 anti-rabbit IgG (1:250, Invitrogen). Cells were washed in PBS and imaged on a Zeiss Axiovert Fluorescence Microscope, 40× objective. All images were processed in the same fashion to remove background in ImageJ by combining all images into a stack and applying subtract background with radius of 50 (images not shown). Data represent the mean+/−standard error from analysis of n>50 cells from at least 4 image fields and are representative of 3 independent experiments. Statistical significance was determined by Mann-Whitney U-test using Graphpad Prism 8.0 software.

These data demonstrate that dual MSC+ HSC treatment significantly enhances expression of Tuj1 following dopaminergic neuronal injury. On the basis of these data, a predominant benefit of dual stem cell therapy is it affords enhanced expression of factors including Tuj1 that are integral to neurite outgrowth, an integral process in dopaminergic neuronal regeneration.

### Example 5—Mechanism of Action: MSCs and HSCs Promote Regeneration Via Both Paracrine Factors and Cell-Cell Interactions

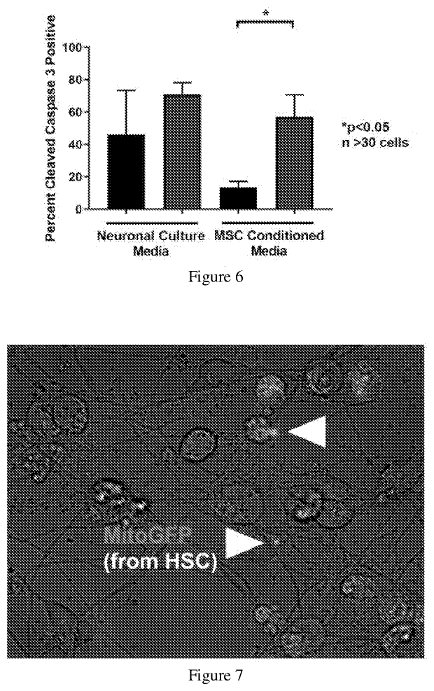

Prior work presented conflicting views of mechanisms of regeneration. Stem cells can secrete factors that can support neuroprotection. FIGS. 6 and 2B show the extent to which paracrine mechanisms by themselves can contribute to regeneration. Specifically, it was investigated whether media collected from cultures of MSCs alone (termed MSC conditioned media or MSC CM) can promote regeneration of neurotoxin-injured neurons. Neurons were first injured by neurotoxin treatment and subsequently cultured in normal neuronal media or MSC conditioned media, and regeneration was measured by cleaved caspase 3 expression in detail below.

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 35 mm #1.5 glass bottom dishes (Cellvis Catalog #D35-20-1.5-N) in fresh differentiation media. On Day 6 of differentiation, cells were treated with 100 uM 6-OHDA for 45 minutes, washed and co-cultured with UCB MSC and/or CD133+ HSC at 10:1:LUHMES:MSC+ HSC ratio and incubated for 72 hours. After 3 days, cells were then processed for caspase 3/Tuj1 co-staining. Cells were fixed in 2% paraformaldehyde for 15 minutes at room temperature. Cells were washed in PBS, stained overnight at 4 degrees C. with rabbit Cleaved Caspase 3 antibody (Cell Signaling Technologies, 1:400) and mouse Tuj1 antibody (Biolegend, 1:400) in 0.1% saponin, 0.1% BSA/PBS pH 7.4. Cells were washed in PBS and stained for 1 hour at room temperature with Alexa Fluor 488 anti-mouse IgG and Alexa Fluor 647 anti-rabbit IgG (1:250, Invitrogen). Cells were washed in PBS and imaged on a Zeiss Axiovert Fluorescence Microscope equipped with Apotome.2, 60× oil immersion objective (images not shown). Data represent the mean+/−standard deviation of the mean from analysis of n>50 cells from at least 4 image fields from one representative of 3 independent experiments. Statistical significance was determined by Mann-Whitney U-test using Graphpad Prism 8.0 software.

MSCs can secrete a mixture of proteins (termed MSC conditioned media) that promote regeneration. However, interestingly, MSC conditioned media by itself (i.e. in the absence of MSCs themselves) is not sufficient to fully regenerate neurons after 6-OHDA treatment. Neurons treated with 6-OHDA in MSC conditioned media show lower levels of caspase 3 compared to 6-OHDA treated neurons in the absence of MSC media, however not as low as in untreated neurons. Moreover, MSC conditioned media does not promote robust neurite outgrowth after injury.

To evaluate the contribution of paracrine and direct cell-contact mechanisms to regeneration, regeneration (reduction of cleaved caspase 3 expression and increased neurite outgrowth) after injured neurons were co-cultured directly with stem cells vs. using indirect transwell co-cultures was compared. In transwell co-cultures, stem cells are separated from neurons by a 0.2 um pore membrane that allows the transfer of secreted paracrine molecules, but prevents direct cell contacts. In a head-to-head comparison, MSCs co-cultured directly with LUHMES cells (allowing direct cell-cell contacts) supported significantly enhanced regeneration compared to MSCs indirectly co-cultured in transwells (allowing only paracrine/secreted factors). These data support the assertion that direct cell-cell contacts play a more prominent role in regeneration than indirect paracrine mechanisms.

### Example 6—Mechanism of Action Underlying Neuronal Regeneration Mediated by Direct Cell-Cell Interactions is TNT

MSC mitochondrial transfer via tunneling nanotubules (TNTs) is the significant mechanism of action underlying dopaminergic neuronal regeneration. HSC can also transfer mitochondria to reduce dopaminergic neuronal injury, a mechanism never previously described in HSCs and only observed in MSCs.

In the presence of injured dopaminergic neurons, MSCs extrude TNTs to facilitate mitochondrial transfer (data not shown). LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 35 mm #1.5 glass bottom dishes (Cellvis Catalog #D35-20-1.5-N) in fresh differentiation media. On Day 6 of differentiation, cells were treated with indicated concentrations of 6-OHDA for 45 minutes, washed and co-cultured with PKH26/Mitotracker Green co-labeled UCB MSC at 3:1 LUHMES:MSC ratio and incubated for 24 hours. Z-stack images of cells were collected on a Zeiss Axiovert Fluorescence Microscope, 60× oil immersion objective.

For PKH26/Mitotracker Green co-labeling, MSCs were washed in PBS and stained with PKH26 using PKH26 staining kit (Sigma Aldrich). MSCs were resuspended in 0.1 mL diluent C. 2 ul of PKH26 dye was diluted in 1 mL diluent C, and 0.1 mL was added to MSCs. After 5 minutes, labeling was quenched by addition of MSC media (10% HS in IMDM complete media). Cells were washed twice in MSC media and resuspended in LUHMES complete media. Subsequently, cells were labeled with 200 nM Mitotracker Green for 30 minutes at 37 degrees C. Cells were washed twice in complete LUHMES media and resuspended in LUHMES differentiation media. MSCs were then co-cultured with LUHMES cells at indicated ratios.

Surprisingly, HSC also transfer mitochondria to injured dopaminergic neurons via TNTs, as shown by FIG. 7. Genetically modified HSCs engineered to express a fluorescent mitochondrial protein (MitoGFP) that circumvents concerns with dye leakage was generated. An experiment was performed to assess whether stem cell-derived MitoGFP-labeled mitochondria are transferred to injured neurons during stem cell-neuronal co-cultures.

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 35 mm #1.5 glass bottom dishes (Cellvis Catalog #D35-20-1.5-N) in fresh differentiation media. On Day 6 of differentiation, cells were treated with indicated concentrations of 6-OHDA for 45 minutes, washed and co-cultured with MitoGFP labeled UCB stem cells at 3:1 LUHMES:stem cell ratio in the presence or absence of 350 nM cytochalasin B and incubated for 24 hours. Cells were then imaged on a Zeiss Axiovert Fluorescence Microscope, 60× oil immersion objective. Z-stack images of cells were collected on a Zeiss Axiovert Fluorescence Microscope, 60× oil immersion objective.

For MitoGFP protein expression in stem cells, UCB CD133+ HSC were transduced with MitoGFP lentivirus. Lentivirus was produced by three plasmid transfection of HEK293T cells with pLYS-MitoGFP (Addgene) and lentiviral envelope and packaging plasmids (Dharmacon), and collected and concentrated using Lenti-X Concentrator. Virus was centrifuged and resuspended in DMEM media, and stored at −80 degrees C. until use. Upon virus generation, stem cells were transduced with MitoGFP lentivirus. Cells were thawed and rested for 24 hours prior to transduction. Subsequently, 100,000 viable trypan blue negative cells suspended in 100 ul of complete HSC media were transferred to a round bottom 96 well plate (Costar). MitoGFP lentivirus was added to stem cells and cells were transduced by spinoculation. Plates were spun at 932×g for 2 hours at room temperature. Subsequently, 100 ul of complete media was added dropwise to each well and cells were placed in the tissue culture incubator. After 24 hours, MitoGFP expression was assessed by fluorescence microscopy and fluorescence was observed in approximately 5% of cells. Cells were treated with 1 ug/ml puromycin to select for cells harboring MitoGFP lentivirus which carries a puromycin resistance gene that confers resistance to puromycin-induced cell death. After 24-48 hours of puromycin selection, the proportion of MitoGFP+ cells was increased to approximately 20%, and cells were utilized in co-culture experiments.

HSC-derived mitochondria, labeled using a genetically encoded mitochondrial fluorescent protein that localizes to mitochondria, could be detected in injured neurons. HSCs indeed transfer mitochondria to injured neurons. HSC transfer of mitochondria to injured neurons has never previously been observed or published.

Mitochondrial transfer via tunneling nanotubes (TNTs) via direct cell-cell interactions is a critical mechanism of action of dual MSC+ HSC in regeneration of injured dopaminergic neurons. Since mitochondrial dysfunction is a key underlying mechanism of injury, it was reasoned that mitochondrial transfer may be actively induced by signals from injured neurons to obtain healthy mitochondria from dual MSC+ HSC and restore mitochondrial function as a key mechanism of regeneration. An experiment was performed to determine whether mitochondrial transfer is specifically triggered by neuronal injury by comparing mitochondrial transfer to healthy vs. 6-OHDA-injured dopaminergic neurons.

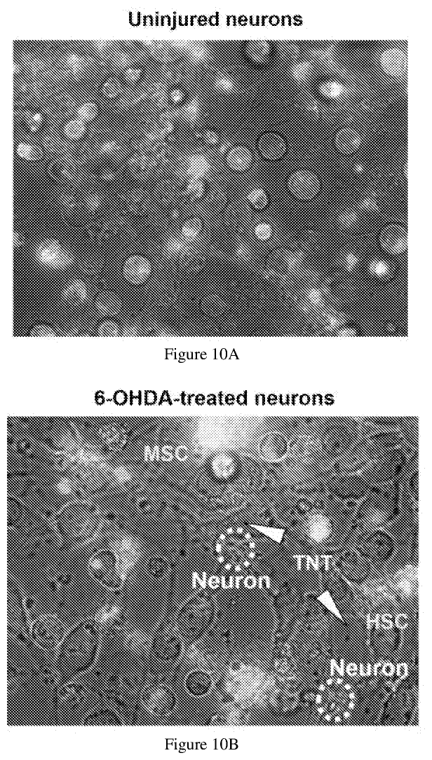

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 35 mm #1.5 glass bottom dishes (Cellvis Catalog #D35-20-1.5-N) in fresh differentiation media. On Day 6 of differentiation, cells were treated with indicated concentrations of 6-OHDA for 45 minutes, washed and co-cultured with Wheat Germ Agglutinin Oregon 488/200 nM Mitotracker Red co-labeled UCB MSC and HSC at 3:1 LUHMES:MSC+ HSC ratio and incubated for 4 hours. MSC and HSC were labeled with 200 nM Mitotracker Red in PBS for 15 minutes. During the final 5 minutes, 1 ug/ml Wheat Germ Agglutinin Oregon 488 was added. Cells were then washed twice in MSC media and co-cultured with neurons for 4 hours. Cells were subsequently imaged on a Zeiss Axiovert Fluorescence Microscope, 60× oil immersion objective (FIGS. 10A-10B).

These experiments show that neuronal injury is required to induce TNT formation and transfer of mitochondria from MSCs and HSCs to dopaminergic neurons. These data demonstrate that TNT formation and mitochondrial transfer from dual MSC+ HSC is an active process that requires signals transmitted by injured dopaminergic neurons to MSC and HSC.

### Example 7—ROS Accumulation in Injured Dopaminergic Neurons is a Major Mechanism that Triggers TNT Formation and Mitochondrial Transfer

Mitochondrial transfer by direct cell-cell interactions is an active process that requires activating mitochondrial pathways to call TNTs carrying mitochondria to them from MSC and HSC. Surprisingly, upregulation of ROS in the injured neurons is the activating event that initiates extrusion of TNTs from therapeutic cells and transfer of mitochondria. Supportive of this mechanism, co-culture of injured neurons with MSC and HSC reduces ROS in injured dopaminergic neurons.

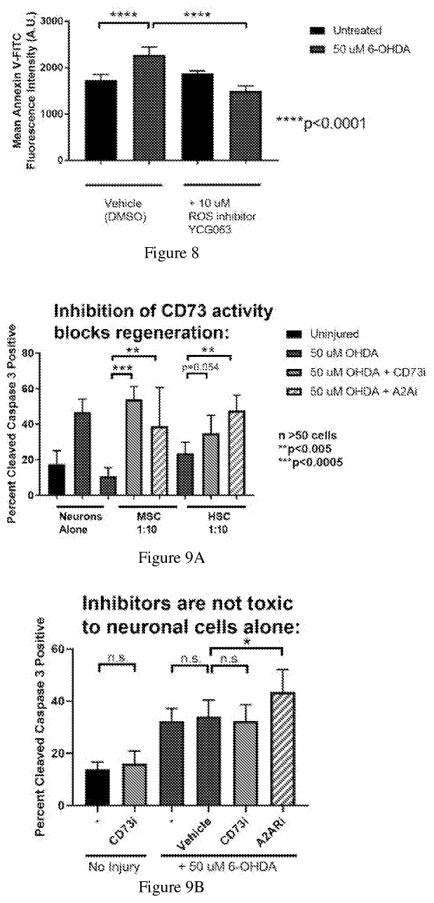

FIG. 8 shows that Blocking ROS reduces 6-OHDA induced injury. ROS accumulation is a key early event during 6-OHDA induced injury of dopaminergic neurons. The following experiment was conducted to determine if ROS accumulation contributes to neuronal injury. In this experiment, whether YCG063, an inhibitor of ROS production, blocks 6-OHDA induced injury, as measured by Annexin V-staining of injured neurons by microscopy was examined.

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 24 well plates in fresh differentiation media. On Day 6 of differentiation, cells were treated 50 uM 6-OHDA in the presence or absence of ROS inhibitor for 45 minutes, washed and cultured as indicated for 3 days. Cells were then stained with Annexin V-FTTC (1:10, BD Biosciences) and Propidium Iodide (1:10, BD Pharmingen) and imaged on a Zeiss Axiovert Fluorescence Microscope, 41× objective (images not shown). Data represent the mean+/−standard deviation from analysis of n>50 cells from at least 4 image fields from 1 experiment.

Inhibition of 6-OHDA induced ROS in dopaminergic neurons significantly reduced 6-OHDA induced neuronal injury measured by Annexin V-staining. These data demonstrate that ROS accumulation is a key mechanism underlying 6-OHDA induced injury of dopaminergic neurons.

ROS accumulation is a key early event during 6-OHDA induced injury of dopaminergic neurons. An experiment was conducted to determine if ROS accumulation contributes to neuronal injury. In this experiment, whether YCG063, an inhibitor of ROS production, blocks 6-OHDA induced injury, as measured by Annexin V-staining of injured neurons by microscopy was examined, as shown by FIG. 11.

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 24 well plates in fresh differentiation media. On Day 6 of differentiation, cells were treated 50 uM 6-OHDA in the presence or absence of ROS inhibitor for 45 minutes, washed and cultured as indicated for 3 days. Cells were then stained with Annexin V-FITC (1:10, BD Biosciences) and Propidium Iodide (1:10, BD Pharmingen) and imaged on a Zeiss Axiovert Fluorescence Microscope, 41× objective (images not shown). Data represent the mean+/−standard deviation from analysis of n>50 cells from at least 4 image fields from 1 experiment.

Inhibition of 6-OHDA induced ROS in dopaminergic neurons significantly reduced 6-OHDA induced neuronal injury measured by Annexin V-staining. These data demonstrate that ROS accumulation is a key mechanism underlying 6-OHDA induced injury of dopaminergic neurons.

### Example 8—Identification of the Major Mechanism Underlying Beneficial Effects of Dual Stem Cells on Regeneration of Injured PD Dopaminergic Neurons Mediated by Direct Cell-Cell Contact

The prior series of experiments indicated that direct cell-cell contacts play a more critical role in stem cell regenerative mechanisms than paracrine mechanisms. Moreover, that an increase in neuronal ROS contributes to neuronal injury was found. It has been reported that increased ROS can serve as a signal to MSCs that stimulates the transfer of healthy mitochondria from MSCs to injured cell types, including neurons. This mechanism may play a critical role particularly in regeneration of injured dopaminergic neurons, in which mitochondrial dysfunction is a key underlying mechanism of injury. Mitochondrial transfer between MSCs and injured cell types occurs in large part via the formation of thin tunneling nanotubes (TNTs) that can be observed by microscopy. That MSCs can transfer fluorescently labeled mitochondria to injured neurons via TNT formation was shown. The following experiments were performed to determine whether CD73 signaling is a critical signal for TNT formation and mitochondrial transfer, utilizing an inhibitor of CD73 signaling.

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 35 mm #1.5 glass bottom dishes (Cellvis Catalog #D35-20-1.5-N) in fresh differentiation media. On Day 6 of differentiation, cells were treated with indicated concentrations of 6-OHDA for 45 minutes, washed and co-cultured with PKH26/Mitotracker Green co-labeled UCB MSC at 3:1 LUHMES:MSC ratio in the presence or absence of CD73 inhibitor (50 uM) and incubated for 8 hours. PKH26 labeling of MSC and HSC was performed as described earlier. Subsequently, MSC and HSC were labeled with 500 nM Mitotracker Green in PBS for 15 minutes. Cells were then washed twice in MSC media and incubated for 4 hours. Cells were subsequently imaged on a Zeiss Axiovert Fluorescence Microscope, 60× oil immersion objective (images not shown). Transfer of MSC/HSC Mitotracker Green-labeled mitochondria to injured neurons was assessed by measuring the mitotracker green fluorescence intensity for individual neuronal cells in ImageJ. Data represent the mean+/−S.D.

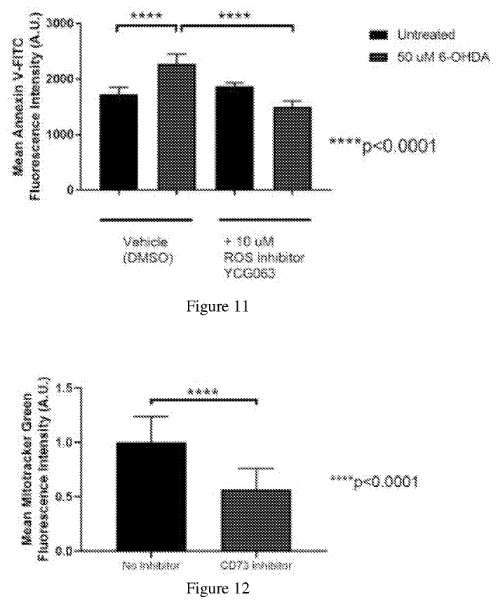

These experiments show that inhibition of CD73 signaling significantly blocks TNT formation and reduces transfer of mitochondria from MSCs and HSCs to injured dopaminergic neurons, as shown by FIG. 12. These data demonstrate that CD73 signaling is a critical pathway that is required for TNT formation and mitochondrial transfer to injured dopaminergic neurons.

Mitochondrial transfer is an active process that requires activating mitochondrial pathways to call TNTs carrying mitochondria to them from MSC and HSC. As previously shown, ROS accumulation in injured neurons is surprisingly the activating event that initiates extrusion of TNTs from therapeutic cells and transfer of mitochondria. Supportive of this mechanism, it was shown that co-culture of injured neurons with MSC and HSC reduces ROS in injured dopaminergic neurons.

ROS induces mitochondrial dysfunction and alters mitochondrial signaling pathways including CD73. CD73 signaling is linked to re-organization of the actin cytoskeleton. The invention discloses that CD73 activation is critical for TNT formation and mitochondrial transfer. This invention surprisingly identified that upregulation of ROS impacts mitochondrial signaling pathways including significant upregulation of CD73 in injured dopaminergic neurons. Further, the invention surprisingly shows that CD73 mediates extrusion of TNTs and mitochondrial transfer that contribute to dopaminergic neuronal regeneration. Surprisingly inhibition of CD73 reduces mitochondrial transfer by 50%. Further, the invention identifies that CD73 inhibition blocks regeneration of injured dopaminergic neurons by MSC and HSC as measured by caspase activation and neurite outgrowth.

The following experiment was carried out to examine whether the CD39 and CD73 proteins, which have been implicated as inflammatory triggers that play protective roles, are upregulated in neurons in multifactorial injury models of Parkinson's disease (after hypoxia or 6-OHDA induced injury) (data not shown).

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×105 cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin 24 well plates in fresh differentiation media. For injury by 6-OHDA, On Day 6 of differentiation, cells were treated with 50 uM 6-OHDA for 45 minutes, washed and incubated for 4 or 24 hours. For injury by hypoxia, 24 well plates are placed in a hypoxia chamber (C-Chamber, Biospherix) and cells are exposed to 5% oxygen for 24 hours delivered using a custom gas mixture consisting of 5.25% carbon dioxide and 94.75% Nitrogen delivered using an oxygen controller (P15 O2 controller, Biospherix). After 4 or 24 hours, plates were removed from hypoxia chamber. All samples were then processed for flow cytometry staining as follows: LUHMES neurons are dissociated from 24 well plates using 0.25% trypsin/EDTA for 5 minutes in the tissue culture incubator. Cells were then rinsed to detach them from the surface using a 5 ml pipet, and mixed with warm complete DMEM/F12 media. Cells were then centrifuged at 14000 rpm for 5 minutes and for each condition, 100,000 cells are resuspended with P1000 to disrupt cell clumps in FACS buffer (0.1% BSC/PBS) and stained using human CD39 PE-CF594 or CD73 PE-Cy7 antibody (BD) for 30 minutes on ice. Cells were washed twice in FACS buffer, resuspended in FACS buffer and 10000 events per tube analyzed utilizing the BD Fortessa flow cytometer (Cleveland Clinic). Data was analyzed in FACS analyzer software (data not shown).

This experiment shows that CD73 is upregulated after both hypoxic and toxin-induced injury of dopaminergic neurons, and triggers TNT extrusion and mitochondrial transfer from adjacent MSC and HSC. The role of CD73 in activating TNT extrusion and transfer of mitochondria to injured dopaminergic neurons has not previously been observed or published.

This invention further shows that CD73 mediates regeneration of injured dopaminergic neurons. The findings from the experiment described above demonstrated that stem cell upregulate CD73 on injured neurons. The following experiment was carried out to examine whether CD73 or A2A receptor activity is involved in MSC or HSC-mediated regeneration. To this end, whether inhibitors of CD73 or A2A receptor activity block regeneration of neurons, as measured by cleaved caspase 3 expression was evaluated.

LUHMES cells were maintained for less than 10 passages in T25 tissue culture flasks pre-coated with 50 ug/ml poly-L-ornithine (Sigma) and 1 ug/ml fibronectin (Sigma) in Complete DMEM/F12 media (Gibco) (containing 1×N-2 Supplement (Gibco), 100 U/ml Penicillin/Streptomycin and 40 ng/ml human basic FGF (Sigma Aldrich)). For experiments, LUHMES cells were dissociated using TrypLE and plated at 7×10⁵ cells per well in a poly-ornithine/fibronectin pre-coated 6 well tissue culture treated plate. The next day, the media was replaced with differentiation media (Complete DMEM/F12 culture media lacking bFGF, supplemented with 1 mM dibutyryl cyclic AMP (Sigma), 1 ug/ml tetracycline (Sigma) and 2 ng/ml glial-derived neurotrophic factor (R&D Systems)). On Day 2 of differentiation, cells were re-plated at 300,000 cells per well to poly-ornithine/fibronectin pre-coated 24 well plates in fresh differentiation media. On Day 6 of differentiation, cells were treated with indicated concentrations of 6-OHDA for 45 minutes, washed and co-cultured with PKH26-labeled UCB MSC and UCB CD133+ HSC at indicated ratios and incubated for 72 hours. Cells were then fixed in 2% paraformaldehyde for 15 minutes at room temperature. Cells were washed in PBS, stained overnight at 4 degrees C. with rabbit Cleaved Caspase 3 antibody (Cell Signaling Technologies, 1:400) and mouse Tuj1 antibody (Biolegend, 1:400) in 0.1% saponin, 0.1% BSA/PBS pH 7.4. Cells were washed in PBS and stained for 1 hour at room temperature with Alexa Fluor 488 anti-mouse IgG and Alexa Fluor 647 anti-rabbit IgG (1:250, Invitrogen). Cells were washed in PBS and imaged on a Zeiss Axiovert Fluorescence Microscope, 40× objective (images not shown). All images were processed in the same fashion to remove background in ImageJ by combining all images into a stack and applying subtract background with radius of 50 and despeckle to remove noise. Data represent the mean+/−standard deviation of the mean from analysis of n>50 cells from at least 4 image fields from 1 experiment. Statistical significance was determined by Mann-Whitney U-test using Graphpad Prism 8.0 software.

For PKH26 labeling, MSCs were washed in PBS and stained with PKH26 using PKH26 staining kit (Sigma Aldrich). MSCs were resuspended in 0.1 mL diluent C. 2 ul of PKH26 dye was diluted in 1 mL diluent C, and 0.1 mL was added to MSCs. After 5 minutes, labeling was quenched by addition of MSC media (10% HS in IMDM complete media). Cells were washed twice in MSC media and resuspended in LUHMES differentiation media. MSCs were then co-cultured with LUHMES cells at indicated ratios.

Inhibitors of CD73 and A2A receptor each dramatically impaired regeneration by either MSC or HSC, as summarized by FIGS. 9A-9B. These data show that the major mechanism of adoptive cell MSC/HSC neuro-regeneration requires direct cell-cell interactions and is not mediated via paracrine mechanism to any major degree. Further, the major mechanism of neuronal regeneration via direct cell-cell interactions is not a passive event but rather active, and further that the signaling pathways that initiate TNT extrusion and mitochondrial transfer from MSC/HSC includes immediate upregulation of ROS in injured dopaminergic neurons and activation of CD73 and A2A receptors.

The invention provides MSC and HSC-mediated partial restoration of dopaminergic function and motor deficits via 1) synergistic neurotrophic and angiogenic effects of MSCs and HSCs 2) combining stem cell therapy with DBS.

The invention provides that combined administration of MSCs and HSCs benefits neuronal survival in vitro, establishing them as a viable preclinical cell therapeutic candidate. The invention provides combined MSC/HSC treatment as a way to enhance neuroprotection in an in vitro model system of PD dopaminergic cell damage. As shown in the data and figures described, the model neurotoxin 6-OHDA induces activation of caspase 3, an early injury marker, and compromises the structural integrity of neurites in LUHMES neuronal cells. The addition of UCB MSCs after neuronal injury markedly reduces neuronal caspase activation and enhances preservation of neurite structure. Nevertheless, MSCs alone cannot completely abrogate neuronal injury. Excitingly, however, the addition of HSCs together with MSCs dramatically enhances neuronal protection. Regeneration is both via paracrine mechanism and direct cell-cell interactions. Direct cell-cell interactions are via TNT and mitochondrial transfer from MSC and HSC to neurons. ROS is a major mechanism driving TNT formation and mitochondrial transfer. CD73 signaling mediates TNT formation, mitochondrial transfer, and neuronal regeneration.

### REFERENCES

1. Wright Willis, A., Evanoff, B. A., Lian, M., Criswell, S. R. & Racette, B. A. Geographic and ethnic variation in Parkinson disease: a population-based study of US Medicare beneficiaries. Neuroepidemiology 34, 143-151 (2010).
2. Parkinson.org. National Parkinson Foundation—Understanding Parkinson's: Causes and Statistics. [online] Available at:http://www.parkinson.org/Understanding-Parkinsons/Causes-and-Statistics—[Accessed 10 Oct. 2018]. (2018).
3. Poewe, W., et al. Parkinson disease. Nature reviews. Disease primers 3, 17013 (2017).
4. Bjorklund, A. & Lindvall, O. Replacing Dopamine Neurons in Parkinson's Disease: How did it happen? Journal of Parkinson's disease 7, S21-S31 (2017).
5. Fu, M. H., et al. Stem cell transplantation therapy in Parkinson's disease. SpringerPlus 4, 597 (2015).
6. Nagamura-Inoue, T. & He, H. Umbilical cord-derived mesenchymal stem cells: Their advantages and potential clinical utility. World journal of stem cells 6, 195-202 (2014).
7. Jordens, C. F., et al. Religious perspectives on umbilical cord blood banking. Journal of law and medicine 19, 497-511 (2012).
8. Gugliandolo, A., Bramanti, P. & Mazzon, E. Mesenchymal stem cell therapy in Parkinson's disease animal models. Current research in translational medicine 65, 51-60 (2017).
9. Yasuhara, T., Kameda, M., Sasaki, T., Tajiri, N. & Date, I. Cell Therapy for Parkinson's Disease. Cell transplantation 26, 1551-1559 (2017).
10. Trounson, A. & McDonald, C. Stem Cell Therapies in Clinical Trials: Progress and Challenges. Cell stem cell 17, 11-22 (2015).
11. Takakura, N., et al. A role for hematopoietic stem cells in promoting angiogenesis. Cell 102, 199-209 (2000).
12. Finney, M. R., et al. Direct comparison of umbilical cord blood versus bone marrow-derived endothelial precursor cells in mediating neovascularization in response to vascular ischemia. Biology of blood and marrow transplantation: journal of the American Society for Blood and Marrow Transplantation 12, 585-593 (2006).
13. Finney, M. R., et al. Umbilical cord blood-selected CD133(+) cells exhibit vasculogenic functionality in vitro and in vivo. Cytotherapy 12, 67-78 (2010).
14. Bhakta, S. & Laughlin, M. J. New developments with umbilical cord blood. Cytotherapy 10, 105-107 (2008).
15. Goldberg, J. L. & Laughlin, M. J. UC blood hematopoietic stem cells and therapeutic angiogenesis. Cytotherapy 9, 4-13 (2007).
16. Goldberg, J. L., Laughlin, M. J. & Pompili, V. J. Umbilical cord blood stem cells: implications for cardiovascular regenerative medicine. Journal of molecular and cellular cardiology 42, 912-920 (2007).
17. Greco, N. & Laughlin, M. J. Umbilical cord blood stem cells for myocardial repair and regeneration. Methods in molecular biology 660, 29-52 (2010).
18. Tse, W., Bunting, K. D. & Laughlin, M. J. New insights into cord blood stem cell transplantation. Current opinion in hematology 15, 279-284 (2008).
19. Tse, W. & Laughlin, M. J. Umbilical cord blood transplantation: a new alternative option. Hematology. American Society of Hematology. Education Program, 377-383 (2005).
20. Inden, M., et al. Therapeutic effects of human mesenchymal and hematopoietic stem cells on rotenone-treated parkinsonian mice. Journal of neuroscience research 91, 62-72 (2013).
21. Lu, J., et al. Hematopoietic stem cells improve dopaminergic neuron in the MPTP-mice. Frontiers in bioscience 18, 970-981 (2013).
22. van Home, C. G., et al. Peripheral nerve grafts implanted into the substantia nigra in patients with Parkinson's disease during deep brain stimulation surgery: 1-year follow-up study of safety, feasibility, and clinical outcome. Journal of neurosurgery, 1-12 (2018).
23. Rowland, N. C., et al. Combining cell transplants or gene therapy with deep brain stimulation for Parkinson's disease. Movement disorders: official journal of the Movement Disorder Society 30, 190-195 (2015).
24. Rowland, N. C., et al. Merging DBS with viral vector or stem cell implantation: “hybrid” stereotactic surgery as an evolution in the surgical treatment of Parkinson's disease. Molecular therapy. Methods & clinical development 3, 15051 (2016).
25. Frenette, P. S., Pinho, S., Lucas, D. & Scheiermann, C. Mesenchymal stem cell: keystone of the hematopoietic stem cell niche and a stepping-stone for regenerative medicine. Annual review of immunology 31, 285-316 (2013).
26. Thanvi, B., Lo, N. & Robinson, T. Vascular parkinsonism—an important cause of parkinsonism in older people. Age and ageing 34, 114-119 (2005).
27. Gupta, D. & Kuruvilla, A. Vascular parkinsonism: what makes it different? Postgraduate medical journal 87, 829-836 (2011).
28. van der Holst, H. M., et al. Cerebral small vessel disease and incident parkinsonism: The RUN DMC study. Neurology 85, 1569-1577 (2015).
29. Laughlin, M. J. Umbilical cord blood for allogeneic transplantation in children and adults. Bone marrow transplantation 27, 1-6 (2001).
30. Laughlin, M. J., et al. Hematopoietic engraftment and survival in adult recipients of umbilical-cord blood from unrelated donors. The New England journal of medicine 344, 1815-1822 (2001).
31. Laughlin, M. J., et al. Outcomes after transplantation of cord blood or bone marrow from unrelated donors in adults with leukemia. The New England journal of medicine 351, 2265-2275 (2004).
32. Laughlin, M. J., et al. Hematologic engraftment and reconstitution of immune function post unrelated placental cord blood transplant in an adult with acute lymphocytic leukemia. Leukemia research 22, 215-219 (1998).
33. Lotharius, J., et al. Effect of mutant alpha-synuclein on dopamine homeostasis in a new human mesencephalic cell line. The Journal of biological chemistry 277, 38884-38894 (2002).
34. Scholz, D., et al. Rapid, complete and large-scale generation of post-mitotic neurons from the human LUHMES cell line. Journal of neurochemistry 119, 957-971 (2011).
35. Zhang, X. M., Yin, M. & Zhang, M. H. Cell-based assays for Parkinson's disease using differentiated human LUHMES cells. Acta pharmacologica Sinica 35, 945-956 (2014).
36. Harris, G., Hogberg, H., Hartung, T. & Smirnova, L. 3D Differentiation of LUHMES Cell Line to Study Recovery and Delayed Neurotoxic Effects. Current protocols in toxicology 73, 11 23 11-11 23 28 (2017).
37. Smirnova, L., et al. A LUHMES 3D dopaminergic neuronal model for neurotoxicity testing allowing long-term exposure and cellular resilience analysis. Archives of toxicology 90, 2725-2743 (2016).
38. Kim, D. H., et al. Thrombospondin-1 secreted by human umbilical cord blood-derived mesenchymal stem cells rescues neurons from synaptic dysfunction in Alzheimer's disease model. Scientific reports 8, 354 (2018).
39. Acquistapace, A., et al. Human mesenchymal stem cells reprogram adult cardiomyocytes toward a progenitor-like state through partial cell fusion and mitochondria transfer. Stem cells 29, 812-824 (2011).
40. Babenko, V. A., et al. Mirol Enhances Mitochondria Transfer from Multipotent Mesenchymal Stem Cells (MMSC) to Neural Cells and Improves the Efficacy of Cell Recovery. Molecules 23(2018).
41. Chen, J., et al. Umbilical Cord-Derived Mesenchymal Stem Cells Suppress Autophagy of T Cells in Patients with Systemic Lupus Erythematosus via Transfer of Mitochondria. Stem cells international 2016, U.S. Pat. No. 4,062,789 (2016).
42. Cho, Y. M., et al. Mesenchymal stem cells transfer mitochondria to the cells with virtually no mitochondrial function but not with pathogenic mtDNA mutations. PloS one 7, e32778 (2012).
43. Han, H., et al. Bone marrow-derived mesenchymal stem cells rescue injured H9c2 cells via transferring intact mitochondria through tunneling nanotubes in an in vitro simulated ischemia/reperfusion model. Molecular medicine reports 13, 1517-1524 (2016).
44. Lin, H. Y., et al. Mitochondrial transfer from Wharton's jelly-derived mesenchymal stem cells to mitochondria-defective cells recaptures impaired mitochondrial function. Mitochondrion 22, 31-44 (2015).
45. Mahrouf-Yorgov, M., et al. Mesenchymal stem cells sense mitochondria released from damaged cells as danger signals to activate their rescue properties. Cell death and differentiation 24, 1224-1238 (2017).
46. Rodriguez, A. M., Nakhle, J., Griessinger, E. & Vignais, M. L. Intercellular mitochondria trafficking highlighting the dual role of mesenchymal stem cells as both sensors and rescuers of tissue injury. Cell cycle, 1-25 (2018).
47. Wang, J., et al. Cell adhesion-mediated mitochondria transfer contributes to mesenchymal stem cell-induced chemoresistance on T cell acute lymphoblastic leukemia cells. Journal of hematology & oncology 11, 11 (2018).
48. Hayakawa, K., et al. Transfer of mitochondria from astrocytes to neurons after stroke. Nature 535, 551-555 (2016).
49. Du, F., Yu, Q., Chen, A., Chen, D. & Yan, S. S. Astrocytes Attenuate Mitochondrial Dysfunctions in Human Dopaminergic Neurons Derived from iPSC. Stem cell reports 10, 366-374 (2018).
50. Dupont, W. D. & Plummer, W. D., Jr. Power and sample size calculations. A review and computer program. Controlled clinical trials 11, 116-128 (1990).
51. Mukherjee, K., et al. Analysis of Brain Mitochondria Using Serial Block-Face Scanning Electron Microscopy. Journal of visualized experiments: JoVE (2016).

1. A method for treating injured neurons in a subject in need comprising administering to the subject a treatment effective amount of a first composition of substantially purified CD133+ hematopoietic stem cells (HSCs) and a second composition of substantially purified mesenchymal stromal cells (MSCs).

2. The method of claim 1, wherein the first and second compositions are combined prior to administration.

3. The method of claim 2, wherein the HSCs and MSCs are autologous to the subject.

4. The method of claim 2, wherein the HSCs and MSCs are allogeneic to the subject.

5. The method of claim 4, wherein the allogenic HSCs and MSCs are from human umbilical cord blood.

6. The method of claim 1, wherein the substantially purified compositions are at least 80% free from other constituents naturally found in blood.

7. The method of claim 1, wherein the HSCs and MSCs are administered by intra-parenchymal injection via stereotactic guidance into the brain of the subject.

8. The method of claim 7, wherein the neuronal injury is due to Parkinson's disease.

9. The method of claim 8, further comprising electrically stimulating the brain of the subject after the administration.

10. A pharmaceutical composition comprising a therapeutically effective dose of substantially purified CD133+ hematopoietic stem cells (HSCs) combined with substantially purified mesenchymal stromal cells (MSCs).

11. A method for producing a composition for treating a neurological condition comprising:
providing blood;
isolating a substantially pure composition of CD133+ hematopoietic stem cells (HSCs) from the blood;
isolating a substantially pure composition of mesenchymal stromal cells (MSCs) from the blood;
combining the composition of substantially pure CD133+ HSCs and the composition of substantially pure MSCs, to produce a composition for treating a neurological condition.

12. The method of claim 10, wherein the blood is human umbilical cord blood.

13. The method of claim 10, wherein the MSCs are further cultured from mononuclear cells in the blood (MNCs) prior to isolation.

14. The method of claim 10, wherein the substantially purified compositions are at least 80% free from other constituents in the blood.

15. A method for treating a neurological condition in a subject in need thereof comprising administering to the subject an effective amount of the composition of claim 10.

16. The method of claim 15, wherein the administration is by intra-parenchymal injection via stereotactic guidance into the brain of the subject.

17. The method of claim 16, wherein the neuronal injury is due to Parkinson's disease.

18. The method of claim 17, further comprising electrically stimulating the brain of the subject after the administration.

19. The method of claim 11, further comprising promoting tunneling nanotubules formation to promote transfer of mitochondria from HSCs and MSCs to injured neurons in the composition prior to administration.

20. The method of claim 11, further comprising combining an effective amount of a reactive oxygen species with the composition to promote transfer of mitochondria from HSCs and MSCs to injured neurons prior to administration.

21. The method of claim 11, further comprising activating CD73 or A2A signaling in the composition to promote transfer of mitochondria from HSCs and MSCs to injured neurons prior to administration.

22. The method of claim 21, wherein type 1 IFNs, TNFa, IL-1b, prostaglandin (PG) E2, TGF-β, agonists of the wnt signaling pathway, E2F-1, CREB, Sp1, HIF1-a, Stat3, or hypoxia is used to activate CD73 signaling.
